(19) United States
(12) Patent Application Publication
(10) Pub. No.: US 2006/0035883 A1
(43) Pub. Date: Feb. 16, 2006

(54) Novel serotonin receptor ligands and their uses thereof

(76) Inventors: Clark E. Tedford, Poulsbo, WA (US); Raghavan Rajagopalan, Solon, OH (US)

Correspondence Address: Marcia S. Kelbon, Esq.;OMEROS CORPORATION, Suite 2600, 1420 Fifth Avenue, Seattle, WA, 98101, US

(21) Appl. No.: 11/202,628

(22) Filed: Aug. 12, 2005

Related U.S. Application Data
(60) Provisional application No. 60/601,296, filed on Aug. 13, 2004.

(51) Int. Cl. A61K 31/553; A61K 31/55; C07D 498/04; C07D 491/04; C07D 487/04; A61K 31/554

(52) U.S. Cl. 514/211.09; 514/215; 540/546; 540/579

(57) ABSTRACT

The present invention discloses novel 5-HT receptor binding agents of Formula 3, 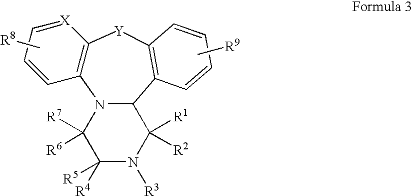
wherein X is —CH or —N—. Y is selected from the group consisting of —CR¹⁰R¹¹, —NR¹², —O—, —S—, —SO—, and —SO₂—. R¹ to R¹² are various substituents selected to optimize the physicochemical and biological properties such as receptor binding, receptor selectivity, tissue penetration, lipophilicity, toxicity, bioavailability, and pharmacokinetics of compounds of Formula 3. These include hydrogen, alkyl, acyl, hydroxyl, hydroxyalkyl, aryl, amino, aminoalkyl, alkoxyl, aryloxyl, carboxyl, alkoxycarbonyl, halogen, cyano, and other suitable electron donating or electron withdrawing groups. R² and R³, R³ and R⁴, or R⁵ and R⁶ may optionally be tethered together to form fused alicyclic or heterocyclic ring. R¹ and R², R⁴ and R⁵, or R⁶ and R⁷ may also optionally be tethered together to form spiro carbo- or heterocyclic ring.

### CROSS REFERENCE TO RELATED APPLICATIONS

This application claims the benefit of the filing date of U.S. Provisional Application No. 60/601,296, filed Aug. 13, 2004.

### FIELD OF THE INVENTION

This invention pertains to novel serotonin (5-HT) receptor antagonists and their potential uses.

### BACKGROUND OF THE INVENTION

Serotonin (5-hydroxytryptamine, 5-HT) is a neurotransmitter that plays a key role in numerous normnal physiological processes such as hemodynamics, feeding, sleeping, etc. as well in as pathophysiological conditions including depression, anxiety, migraine, hypertension, etc. At present, four serotonin receptor subtypes have been identified: 5-HT₁, 5-HT₂, 5-HT₃, and 5-HT₄. The 5-HT₂ receptor family is further subdivided into 5-HT_{2A}, 5-HT_{2B}, and 5-HT_{2C} subtypes. Among the 5-HT₂ family, the 5-HT_{2A} subtype has been extensively studied with respect to its distribution and function, both in the brain and in the periphery [1]. The 5-HT_{2C} receptor subtype is known to be distributed throughout the brain, but not in the peripheral tissues. In contrast, the distribution and function of the 5-HT_{2B} subtype has not yet been well explored. There is a very close structural and functional similarity between 5-HT_{2A} and 5-HT_{2C} subtypes, which suggests that the pharmacological activity once attributed to the 5-HT_{2A} receptor could have been mediated by the 5-HT_{2C} receptor. Indeed, despite the discovery of hundreds of high-affinity serotonin receptor binding ligands over the past several decades, there still is a lack of selectivity; some of the ligands have also shown to cross-react with other receptors such as α₁ adrenergic, and dopamine receptors [2].

Compounds of the present invention may be used to treat a subject suffering from CNS disorders such as schizophrenia, (and other psychotic disorders such as paranoia and mano-depressive illness), Parkinson's disease and other motor disorders, anxiety (e.g. generalized anxiety disorders, panic attacks, and obsessive compulsive disorders), depression (such as by the potentiation of serolonin reuptake inhibitors and serotonin norepinephrine reuptake inhibitors), Tourette's syndrome, migraine, autism, attention deficit disorders and hyperactivity disorders. Compounds of the present invention may also be useful for the treatment of sleep disorders, social phobias, pain, thermoregulatory disorders, endocrine disorders, urinary incontinence, vasospasm, stroke, eating disorders such as for example obesity, anorexia and bulimia, sexual dysfunction, and the treatment of alcohol, drug and nicotine withdrawal.

Compounds of the present invention are also useful for the treatment of cognitive dysfunction. Thus, compounds of the present invention may be useful for the treatment of cognitive dysfunction associated with mild cognitive impairment (MCI)) Alzheimer's disease and other dementias including Lewy Body, vascular, and post stroke dementias. Cognitive dysfunction associated with surgical procedures, traumatic brain injury or stroke may also be treated in accordance with the present invention. Further, compounds of the present invention may be useful for the treatment of diseases in which cognitive dysfunction is a co-morbidity such as, for example, Parkinson's disease, autism and ADHD.

5-HT_{2A} and/or 5-HT_{2C} may also be important in mediating the behavioral actions of psychostimulants. The motor-activating effects of acute cocaine are blocked by intracerebrally injected 5-HT_{2A/2C} antagonists [4]. The discriminative-stimulus effect s of METH and cocaine in monkeys and rats are reduced by 5-HT_{2A/2C} antagonists such as p-chlorophenylalanine, but are potentiated by 5-HT_{2A/2C} agonists such as ±-1-(2,5-dimethoxy-4-iodophenyl)-2-aminopropane (DOI) [5-7]. In human cocaine addicts, the craving normally elicited by environmental stimuli previously associated with cocaine administration is reduced following a reduction in serotonin levels by lowering plasma levels of its precursor, tryptophan, and that the short-term withdrawal from repeated cocaine leads to enhanced behavioral and neuroendocrine responses which are mediated by 5-HT_{2A/2C} receptors [8,9]. This short time course for changes correlates with the negative mood associated with initial abstinence, but drug-craving far outlasts the duration of the initial anhedonic phase.

There is an emerging theme that decreased 5-HT transmission can attenuate the

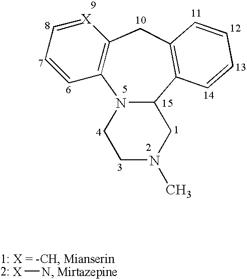

subjective experience and incentive motivation for psychostimulation. Mianserin (1) and mirtazepine (2) are potent 5-HT₂ receptor antagonists that are being currently used as antidepressants. In addition, mianserin has recently been shown to reverse behavioral sensitization due to prior cocaine use, and 5-HT₂ receptor antagonists have been suggested to be a useful treatment for cocaine addicts who have undergone previous sensitization periods [10]. However, as shown in Table 1, these compounds

TABLE 1
Receptor affinities of 5-HT receptor antagonists (Kᵢ in nM).

| Receptor | Mianserin (1) | Mirtazepine (2) |
|---|---|---|
| 5-HT_{1A} | >1000 | >1000 |
| 5-HT_{2A} | 2 | 6 |
| 5-HT_{2C} | 5 | 12 |
| 5-HT₃ | 8 | 8 |
| D₁ | >1000 | >1000 |
| D₂ | >1000 | >1000 |
| H₁ | 2 | 0.5 |
| α₁ | 80 | 500 |
| α₂ | 40 | 65 |
| SERT | >1000 | >1000 |
| NET | 30 | >1000 |

do not exhibit high selectivity for ⁵-HT_{2A/2C} receptors. Receptor selectivity is an important consideration for drug development because side effects of drugs are often attributed to non-selectivity of the ligands. Thus, there is a need in the art to develop potent and very selective 5-HT_{2A/2C} receptor ligands to develop effective drugs to treat numerous normal physiological and pathological processes mediated by serotonin receptors.

In the past several decades, numerous of analogs of mianserin (1) and mirtazepine (2) have been prepared [12, 13], but all of them were derived from the substitutions at the two phenyl rings (positions 6-9 and 11-14), at the central methylene (position 10), or at the piperazine nitrogen (position 2). Also, position 10 has been replaced with oxygen, nitrogen, and sulfur atoms to give the corresponding oxazapine, diazepine, and thiazepine analogs respectively that exhibit potent pharmacological properties. Surprisingly, none of the published works discloses analogs of ligands 1 and 2 wherein positions 1, 3, or 4 have been substituted even with simple alkyl groups such as methyl or ethyl. Since there is a close similarity in the structures of 5-HT subtypes, it is not possible to predict ligand selectivity a priori given the current state of the art with respect to empirical data as well as molecular modeling methods; it is possible that even a subtle changes in the core ligand structure may lead to substantial changes in the selectivity. Thus, the object of the present invention is to explore the core structures 1 and 2 with appropriate substituents in 1, 3, or 4 positions for the purpose of developing highly selective 5-HT receptor ligands.

### SUMMARY OF THE INVENTION

Accordingly, the present invention discloses novel ligands of Formula 3, 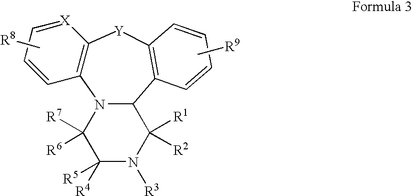
wherein X is —CH or —N—. Y is selected from the group consisting of —CR¹⁰R¹¹, —NR¹², —O—, —S—, —SO—, and —SO₂—. R¹ to R¹² are various substituents selected to optimize the physicochemical and biological properties such as receptor binding, receptor selectivity, tissue penetration, lipophilicity, toxicity, bioavailability, and pharmacokinetics of compounds of Formula 3. These include hydrogen, alkyl, acyl, hydroxyl, hydroxyalkyl, aryl, amino, aminoalkyl, alkoxyl, aryloxyl, carboxyl, alkoxycarbonyl, halogen, cyano, and other suitable electron donating or electron withdrawing groups. R² and R³, R³ and R⁴, or R⁵ and R⁶ may optionally be tethered together to form fused alicyclic or heterocyclic ring. R¹ and R², R⁴ and R⁵, or R⁶ and R⁷ may also optionally be tethered together to form spiro carbo- or heterocyclic ring. The compounds of the present invention may be useful for the treatment of CNS disorders including drug addiction, anxiety, depression, and the like.

### DETAILED DESCRIPTION OF THE INVENTION

The present invention pertains to novel ligands of Formula 3, 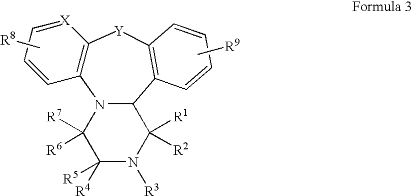
wherein X is —CH or —N—. Y is selected from the group consisting of—CR¹⁰R¹¹, —NR¹², —O—, —S—, —SO—, and —SO₂—. R¹, R², R⁴-R⁷, R¹⁰, and R¹¹ are independently selected from the group consisting of hydrogen; C₁-C₁₀ alkyl; cyano; carboxyl; C₁-C₁₀ acyl; C₁-C₁₀ hydroxyalkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; with the proviso that not all R¹, R², R⁴-R⁷, R¹⁰, and R¹¹ are hydrogens. R³ and R¹² are independently selected from the group consisting of hydrogen; C₁-C₁₀ alkyl; C₁-C₁₀ hydroxyalkyl; C₁-C₁₀ alkxoylcarbonylalkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl. R⁸ and R⁹ are independently selected from the group consisting of hydrogen; C₁-C₁₀ alkyl; hydroxyl, C₁-C₁₀ alkoxyl; C₁-C₁₀ hydroxyalkyl; amino, C₁-C₁₀ alkylamino, C₁-C₁₀ dialkylamino, carboxyl, and C₁-C₁₀ alkxoylcarbonyl. R² and R³, R³ and R⁴, or R⁵ and R⁶ may optionally be tethered together to form fused alicyclic or heterocyclic ring. R¹ and R², R⁴ and R⁵, or R⁶ and R⁷ may also optionally be tethered together to form spiro carbo- or heterocyclic ring.

A preferred embodiment of the present invention is represented by Formula 4, 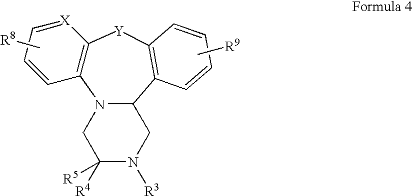
wherein X is —CH or —N—. Y is selected from the group consisting of —CR¹⁰R¹¹, —NR¹², —O—, —S—, —SO—, and —SO₂—. R³ and R¹² are independently selected from the group consisting of hydrogen; C₁-C₁₀ alkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl. R⁴ is selected from the group consisting of C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl. R⁵, R¹⁰, and R¹¹ are independently selected from the group consisting of hydrogen, C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl. R⁸ and R⁹ are independently selected from the group consisting of hydrogen; C₁-C₁₀ alkyl; hydroxyl, C₁-C₁₀ alkoxyl; C₁-C₁₀ hydroxyalkyl; amino, C₁-C₁₀ alkylamino, C₁-C₁₀ dialkylamino, carboxyl, and C₁-C₁₀ alkxoylcarbonyl.

Another preferred embodiment of the present invention is represented by Formula 4, wherein X is —CH or —N—. Y is selected from the group consisting of —CR¹⁰R¹¹, —NR¹², —O—, and —S—. R³ and R¹² are independently selected from the group consisting of hydrogen, C₁-C₁₀ alkyl, phenyl, halophenyl, hydroxyl phenyl, methoxyphenyl, benzyl, hydroxybenzyl, methoxybenzyl, and halobenzyl. R⁴ is selected from the group consisting of C₁-C₁₀ alkyl, phenyl, halophenyl, hydroxyphenyl, methoxyphenyl, benzyl, hydroxybenzyl, methoxybenzyl, and halobenzyl. R⁵, R¹⁰, and R¹¹ are hydrogen. R⁸ and R⁹ are hydrogen or hydroxyl.

Another preferred embodiment of the present invention is represented by Formula 5, 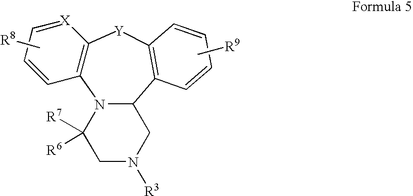
wherein X is —CH or —N—. Y is selected from the group consisting of —CR¹⁰R¹¹, —NR¹², —O—, —S—, —SO—, and —SO₂—. R³ and R¹² are independently selected from the group consisting of hydrogen; C₁-C₁₀ alkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl. R⁶ is selected from the group consisting of C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl. R⁷, R¹⁰, and R¹¹ are independently selected from the group consisting of hydrogen, C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl. R⁸ and R⁹ are independently selected from the group consisting of hydrogen; C₁-C₁₀ alkyl; hydroxyl, C₁-C₁₀ alkoxyl, C₁-C₁₀ hydroxyalkyl, amino, C₁-C₁₀ alkylamino, C₁-C₁₀ dialkylamino, carboxyl, and C₁-C₁₀ alkxoylcarbonyl.

Another preferred embodiment of the present invention is represented by Formula 5, wherein X is —CH or —N—. Y is selected from the group consisting of —CR¹⁰R¹¹, —NR¹², —O—, and —S—. R³ and R¹² are independently selected from the group consisting of hydrogen, C₁-C₁₀ alkyl, phenyl, halophenyl, hydroxyl phenyl, methoxyphenyl, benzyl, hydroxybenzyl, methoxybenzyl, and halobenzyl. R⁶ is selected from the group consisting of C₁-C₁₀ alkyl, phenyl, halophenyl, hydroxyphenyl, methoxyphenyl, benzyl, hydroxybenzyl, methoxybenzyl, and halobenzyl. R⁷, R¹⁰, and R¹¹ are hydrogens. R⁸ and R⁹ are hydrogen or hydroxyl.

Another preferred embodiment of the present invention is represented by Formula 6 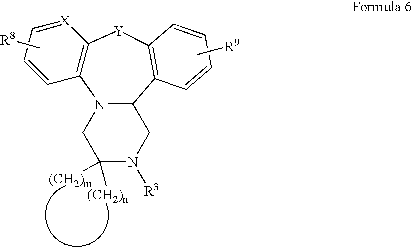
wherein m and n independently vary from 1 to 4. X is —CH or —N—. Y is selected from the group consisting of —CR¹⁰R¹¹, —NR¹², —O—, —S—, —SO—, and —SO₂—. R³ and R¹² are independently selected from the group consisting of hydrogen; C₁-C₁₀ alkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl. R₁₀ and R¹¹ are independently selected from the group consisting of hydrogen, C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl. R⁸ and R⁹ are independently selected from the group consisting of hydrogen; C₁-C₁₀ alkyl; hydroxyl, C₁-C₁₀ alkoxyl; C₁-C₁₀ hydroxyalkyl; amino, C₁-C₁₀ alkylamino, C₁-C₁₀ dialkylamino, carboxyl, and C₁-C₁₀ alkxoylcarbonyl.

Another preferred embodiment of the present invention is represented by Formula 6, wherein m and n independently vary from 1 to 4. X is —CH or —N—. Y is selected from the group consisting of —CR¹⁰R¹¹, —NR¹², —O—, and —S—. R³ and R¹² are independently selected from the group consisting of hydrogen, C₁-C₁₀ alkyl, phenyl, halophenyl, hydroxyphenyl, methoxyphenyl, benzyl, hydroxybenzyl, methoxybenzyl, and halobenzyl. R⁸ and R⁹ are hydrogen or hydroxyl. R¹⁰ and R¹¹ are hydrogen.

Another preferred embodiment of the present invention is represented by Formula 7, 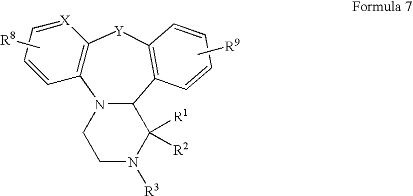
wherein X is —CH or —N—. Y is selected from the group consisting of —CR¹⁰R¹¹, —NR¹², —O—, —S—, —SO—, and —SO₂—. R¹ is selected from the group consisting of C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl. R², R¹⁰, and R¹¹ are independently selected from the group consisting of hydrogen, C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl. R³ and R¹² are independently selected from the group consisting of hydrogen; C₁-C₁₀ alkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; and C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl. R⁸ and R⁹ are independently selected from the group consisting of hydrogen; C₁-C₁₀ alkyl; hydroxyl, C₁-C₁₀ alkoxyl; C₁-C₁₀ hydroxyalkyl; amino, C₁-C₁₀ alkylamino, C₁-C₁₀ dialkylamino, carboxyl, and C₁-C₁₀ alkxoylcarbonyl.

Another preferred embodiment of the present invention is represented by Formula 7, wherein X is —CH or —N—. Y is selected from the group consisting of —CR¹⁰R¹¹, —NR¹², —O—, and —S—. R¹ is selected from the group consisting of C₁-C₁₀ alkyl, phenyl, halophenyl, hydroxyphenyl, methoxyphenyl, benzyl, hydroxybenzyl, methoxybenzyl, and halobenzyl. R², R¹⁰, and R¹¹ are hydrogen. R³ and R¹² are independently selected from the group consisting of hydrogen, C₁-C₁₀ alkyl, phenyl, halophenyl, hydroxyl phenyl, methoxyphenyl, benzyl, hydroxybenzyl, methoxybenzyl, and halobenzyl. R⁸ and R⁹ are hydrogen or hydroxyl.

The compounds belonging to Formula 4 can be synthesized according to the method 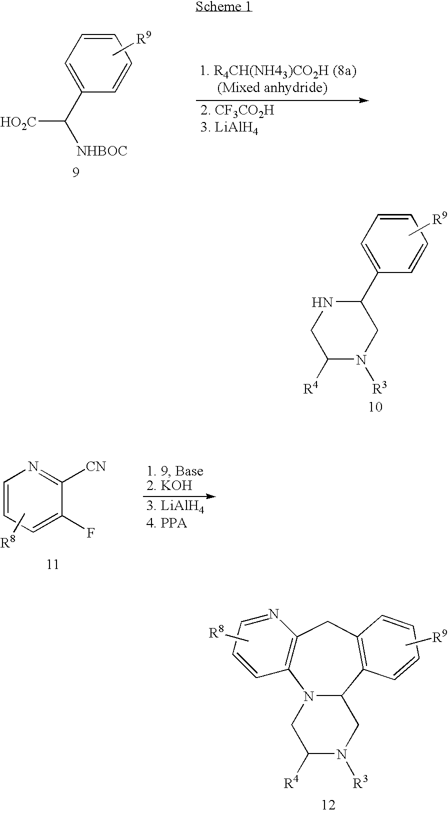
outlined in Scheme 1. The key intermediates, viz., 2, 4, 5-trisubstiuted piperazine derivatives 10, can be prepared by the condensation of two starting materials, N-alkylamino acids 8a and arylglycines 9, in three steps: mixed anhydride coupling of the two amino acids, simultaneous deprotection and ring closure to the diketopiperazine derivative, and the reduction of the diamide with a borane reagent. Some of the arylglycine derivatives are either available commercially (in the case of phenyl glycine) or can be synthesized readily from aromatic aldehydes using the well known Strecker amino acid synthesis. Similarly, some of the N-alkylamino acids are available commercially or can be prepared by diborane reduction of the corresponding t-butyl esters of N-acylamino acids. Further transformation of piperazines 10 to the ligands 12 belonging to the generic Formula 4 can be accomplished by the procedure described in the U.S. patent application [12], incorporated hereby as reference in its entirety.

The compounds belonging to Formula 5 can also be synthesized by the method nearly identical to the one outlined in Scheme 1, except that the acyclic aminoester 8a is replaced with 1-(N-alkyl)aminocycloalkanecarboxylic acid 8b; all other starting materials 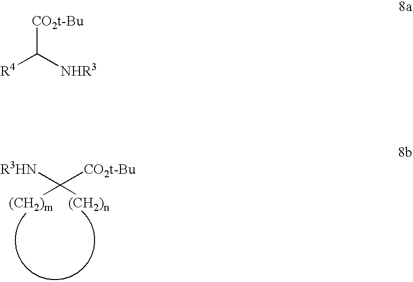
and reagents are identical. Some of the 1-(N-alkyl)aminocyclo-alkanecarboxylic acid derivatives are either available commercially or can be synthesized readily from cycloalkanones using the well known Strecker amino acid synthesis.

The compounds belonging to Formula 6 can be synthesized by the method outlined in Scheme 2. The key intermediates, viz., 2, 4, 6-trisubstiuted piperazine derivatives 15, can 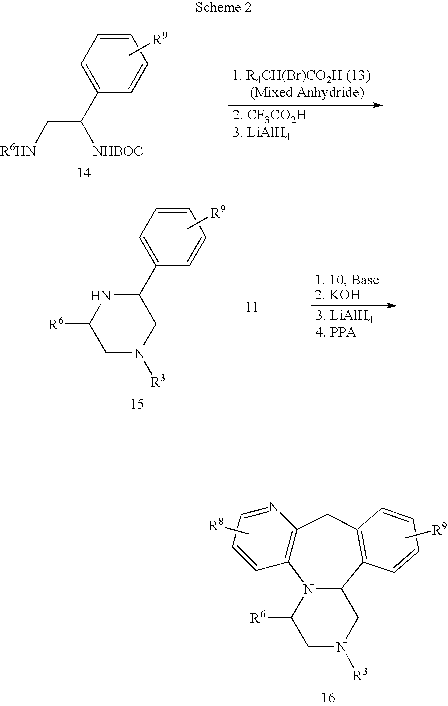
be prepared by the condensation of two starting materials, α-bromocarboxylic acids 13 and N¹-Boc-N²-alkylamino-1-arylethylenediamines 14, in three steps: mixed anhydride coupling of the monoprotected diamine and the α-bromocarboxylic acids, simultaneous deprotection and ring closure to the lactam derivative, and the reduction of the lactam with a borane reagent. The monoprotected diamine 14 can be prepared from N-t-Boc-arylglycines in two steps: mixed anhydride coupling of N-t-Boc-arylglycines with alkylamines followed by the selective reduction of the amide with a borane reagent. Many α-bromocarboxylic acids are available commercially, but they can be also be prepared by bromination of the corresponding carboxylic acids. Conversion of 15 to the ligands 16 belonging to the generic Formula 6 can be accomplished by the procedure identical to the one outlined in Scheme 1.

The compounds belonging to Formula 7 can be synthesized according to the method outlined in Scheme 3. The starting material 17 can be prepared by the method described in 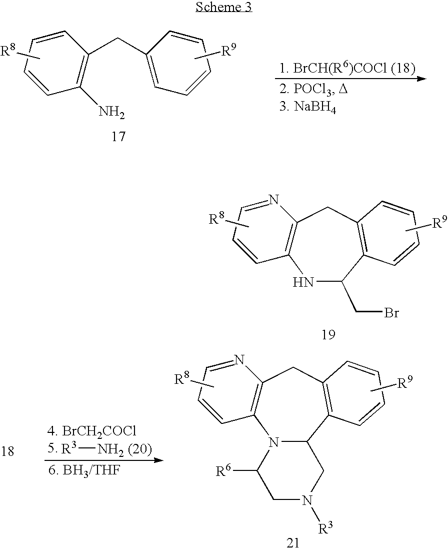
the U.S. Patent [12], incorporated herein by reference. Acylation of the amine in 17 with α-bromoacid chlorides 18 followed by Bischler-Napieralski cyclization of the amide, and reduction of the resulting imine gives the tricyclic amine 19. Acylation of the amines 19 with bromoacetyl chloride, alkylation of the dibromide with the primary amines 20, and reduction of the lactam with either diborane or lithium aluminum hydride gives the ligands 21 belonging to the generic Formula 7.

Compounds of the present invention may exist as a single stereoisomer or as mixture of enantiomers and diastereomers whenever chiral centers are present. Individual stereoisomers can be isolated by the methods well known in the art: diastereomers can be separated by standard purification methods such as fractional crystallization or chromatography, and enantiomers can be separated either by resolution or by chromatography using chiral columns.

The pharmaceutical composition of the present invention may contain the active pharmaceutical ingredient along with physiologically tolerable diluents, carriers, adjuvants, and the like. The phrase “pharmaceutically acceptable” means those formulations which are, within the scope of sound medical judgment, suitable for use in contact with the tissues of humans and animals without undue toxicity, irritation, allergic response and the like, and are commensurate with a reasonable benefit/risk ratio. Pharmaceutically acceptable salts are well-known in the art, and are described by Berge et al. [13], incorporated herein by reference. Representative salts include, but are not limited to acetate, adipate, alginate, citrate, aspartate, benzoate, benzenesulfonate, chloride, bromide, bisulfate, butyrate, camphorate, camphor sulfonate, gluconate, glycerophosphate, hemisulfate, heptanoate, hexanoate, fumarate, maleate, succinate, oxalate, citrate, hydrochloride, hydrobromide, hydroiodide, lactate, maleate, nicotinate, 2-hydroxyethansulfonate (isothionate), methane sulfonate, 2-naphthalene sulfonate, oxalate, palmitoate, pectinate, persulfate, 3-phenylpropionate, picrate, pivalate, propionate, tartrate, phosphate, glutamate, bicarbonate, p-toluenesulfonate, undecanoate, lithium, sodium, potassium, calcium, magnesium, aluminum, ammonium, tetramethyl ammonium, tetraethylammonium, methylammonium, dimethylammonium, trimethylammonium, triethylammonium, diethylammonium, and ethylammonium, and the like.

The pharmaceutical compositions of this invention can be administered to humans and other mammals enterally or parenterally in a solid, liquid, or vapor form. Enteral route includes, oral, rectal, topical, buccal, and vaginal administration. Parenteral route intravenous, intramuscular, intraperitoneal, intrastemal, and subcutaneous injection or infusion. The compositions can also be delivered through a catheter for local delivery at a target site, via an intracoronary stent (a tubular device composed of a fine wire mesh), or via a biodegradable polymer.

The active compound is mixed under sterile conditions with a pharmaceutically acceptable carrier along with any needed preservatives, exipients, buffers, or propellants. Opthalmic formulations, eye ointments, powders and solutions are also contemplated as being within the scope of this invention. Actual dosage levels of the active ingredients in the pharmaceutical formulation can be varied so as to achieve the desired therapeutic response for a particular patient. The selected dosage level will depend upon the activity of the particular compound, the route of administration, the severity of the condition being treated, and prior medical history of the patient being treated. However, it is within the skill of the art to start doses of the compound at levels lower than required to achieve the desired therapeutic effect and to increase it gradually until optimal therapeutic effect is achieved. The total daily dose of the compounds of this invention administered to a human or lower animal may range from about 0.0001 to about 1000 mg/kg/day. For purposes of oral administration, more preferable doses can be in the range from about 0.001 to about 5 mg/kg/day. If desired, the effective daily dose can be divided into multiple doses for purposes of administration; consequently, single dose compositions may contain such amounts or submultiples thereof to make up the daily dose.

The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated, the severity of the disorder; activity of the specific compound employed; the specific composition employed, age, body weight, general health, sex, diet of the patient; the time of administration, route of administration, and rate of excretion of the specific compound employed, and the duration of the treatment. The compounds of the present invention may also be administered in combination with other drugs if medically necessary.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof. These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like. Prolonged absorption of the injectable pharmaceutical form can be brought about by the use of agents delaying absorption, for example, aluminum monostearate and gelatin. Proper fluidity can be maintained, for example, by the use of coating materials such as lecithin, by the maintenance of the required particle size in the case of dispersions, and by the use of surfactants. In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Dosage forms for topical administration include powders, sprays, ointments and inhalants. Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

The present invention also provides pharmaceutical compositions that comprise compounds of the present invention formulated together with one or more non-toxic pharmaceutically acceptable carriers. Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together. Methods to form liposomes are known in the art [xx], incorporated herein by reference.

The compounds of the present invention can also be administered to a patient in the form of pharmaceutically acceptable ‘prodrugs.’ The term “pharmaceutically acceptable prodrugs” as used herein represents those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. Prodrugs of the present invention may be rapidly transformed in vivo to the parent compound of the above formula, for example, by hydrolysis in blood. A thorough discussion is provided by Higuchi and Stella [14], incorporated herein by reference.

The Examples that follow are describe preferred embodiments and utilities of the invention and are not meant to limit the invention unless otherwise stated in the claims. The description is intended as a non-limiting illustration, since many variations will become apparent to those skilled in the art in view thereto. Changes can be made in the composition, operation, and the method of the present invention described herein without departing from the concept and scope of the invention as defined in the claims.

### EXAMPLE 1

### Synthesis of Ligand 22, (Scheme 1. R³=R⁸=R⁹=—H, and R⁴=—CH—)

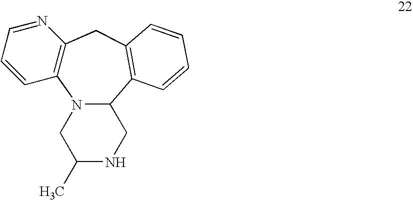

Step 1. A solution of N-Boc-phenylglycine (20 mmol) and triethylamine (50 mmol) in anhydrous methylene chloride (50 mL) is stirred and cooled to 0° C. Isobutylchloroformate (21 mmol) is then added to the mixture at such a rate that the internal temperature was maintained at 0-5° C. The stirring is continued at about 0° C. for 45 minutes. Thereafter, alanine t-butylester (20 mmol) is then added, and the entire mixture is stirred at ambient temperature for 4 hours. The reaction mixture is then poured onto water and the organic layer is separated, washed with 5% hydrochloric acid, saturated sodium bicarbonate, and water. The organic phase is then dried over magnesium sulfate, filtered, and the filtrate evaporated in vacuo to give the protected peptide.

Step 2. The residue from Step 1 is treated with trifluoroacetic acid (TFA)(10 mL), stirred at ambient temperature for 15 minutes, and heated under reflux for 2 hours. The reaction mixture is poured onto water, and extracted with methylene chloride. The organic layer is separated, washed with 5% hydrochloric acid, saturated sodium bicarbonate, and water. The organic phase is then dried over magnesium sulfate, filtered, and the filtrate evaporated in vacuo to give the diketopiperazine interemediate.

Step 3. A solution of the diketopiperazine derivative from Step 2 is in anhydrous tetrahydrofuran (20 mL) is stirred and cooled to 0° C. under inert atmosphere. Thereafter a solution of lithium aluminium hydride (1 M solution in tetrahydrofuran) is carefully added. After the addition, the solution is heated under reflux for 4 hours. Thereafter, the reaction mixture is cooled to 0° C. under inert atmosphere and very carefully treated with water added dropwise to decompose excess reducing agent. After complete decomposition, the solution was treated with anhydrous sodium sulfate, kept at ambient temperature for 1 hour and filtered. The filtrate is evaporated in vacuo to give the crude phenylpiperazine intermediate 9, wherein R³=R⁹=—H, and R⁴=—CH₃. Purification of the intermediate 9 can be accomplished by chromatography or recrystallization.

Step 3-6. The phenylpiperazine intermediate 9 from Step 2 is converted to the final ligand 11 in a 4-step procedure described in the U.S. patent [12], incorporated herein by reference in its entirety. All other R⁴ substituted derivatives can be prepared in an identical manner using other natural d or l amino acid esters such as valine, leucine, phenylalanine, and the like.

### EXAMPLE 2

### Synthesis of Ligand 23, (Scheme 1. R³=R⁴=—CH₃, and R⁸=R⁹=—H)

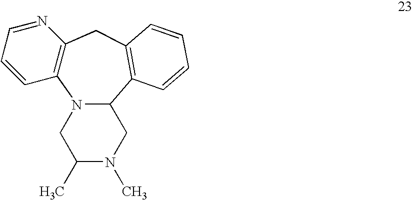

Synthesis of the title compound can be accomplished in an identical manner to the synthesis of the ligand described in Example 1 except that alanine t-butylester is replaced with N-methylalanine t-butylester in Step 1. All other R³ and R⁴ substituted derivatives can be prepared in an identical manner using other natural d or l N-alkylamino acid esters such as N-alkylvaline, N-alkylleucine, N-alkylphenylalanine, and the like.

EXAMPLES 3

### Synthesis of the Spiro Ligand 24, (R⁸=R⁹=—H)

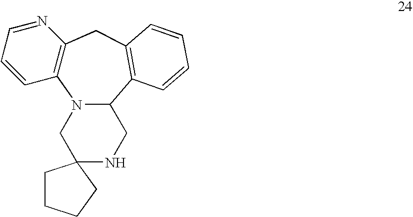

Synthesis of the title compound can be accomplished in an identical manner to the synthesis of the ligand described in Example 1 except that alanine t-butylester is replaced with t-butyl 1-aminocyclopentanecarboxylate in Step 1. All other spiro derivatives can be prepared in an identical manner using other α-amino substituted carbocyclic or heterocyclic carboxylic acids such as 1-aminocyclohexane carboxylic acid, 4-aminopiperdine-4-carboxylic acid, 3-aminotetahydrofuran-3-carboxylic acid, and the like.

### EXAMPLE 5

### Synthesis of Ligand 25, (Scheme 2. R³=R⁸=R⁹=—H, and R⁶=—CH₃)

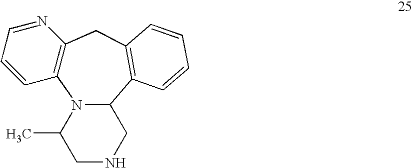

Step 1. A biphasic mixture of N¹-t-Boc-1-phenyl-1,2-diaminoethane (20 mmol) in methylene chloride (50 mL) and sodium carbonate (30 mmol) in water (50 mL) is vigorously stirred and cooled to 10-15° C. in ice bath. Then, 2-bromopropionyl chloride (22 mmol) is then added dropwise to the mixture at such a rate that the internal temperature was maintained at about 15° C. After the addition, the stirring is continued at ambient temperature for about 2 hours. Thereafter, the organic layer is separated, washed with 5% hydrochloric acid, and water. The organic phase is then dried over magnesium sulfate, filtered, and the filtrate evaporated in vacuo to give the protected amide.

Step 2. The residue from Step 1 is treated with trifluoroacetic acid (TFA)(10 mL), stirred at ambient temperature for 30 minutes. The reaction mixture is poured onto water, and extracted with methylene chloride. The organic layer is separated washed with saturated sodium bicarbonate followed by brine, dried over sodium sulfate, filtered, and the filtrate evaporated in vacuo to give the bromoamine intermediate.

Step 3. A mixture of the bromoamine derivative (10 mmol) from Step 2 and finely-ground anhydrous potassium carbonate (20 mmol) in glyme (20 mL) is stirred and heated under reflux for 4 hours. Thereafter, the reaction mixture is filtered hot and the filtrate evaporated in vacuo to give the crude lactam, which is purified by chromatography or recrystallization, or may be used as such in the next step.

Step 4. A solution of the lactam derivative (10 mmol) from Step 3 is in anhydrous tetrahydrofuran (20 mL) is stirred and cooled to 0° C. under inert atmosphere. Thereafter a solution of lithium aluminium hydride (1 M in THF, 10 mL) is carefully added. After the addition, the solution is heated under reflux for 4 hours. Thereafter, the reaction mixture is cooled to 0° C. under inert atmosphere and very carefully treated with water added dropwise to decompose excess reducing agent. After complete decomposition, the solution was treated with anhydrous sodium sulfate, kept at ambient temperature for 1 hour and filtered. The filtrate is evaporated in vacuo to give the crude phenylpiperazine intermediate 14, wherein R⁷=R⁹=—H, and R⁶=—CH₃. Crude intermediate 14 is purified by chromatography or recrystallization.

Step 5-8. The phenylpiperazine intermediate 14 from Step 4 is converted to the final ligand 15 in a 4-step procedure described in the U.S. patent [12], incorporated herein by reference in its entirety.

### EXAMPLE 6

### Synthesis of Ligand 26, (Scheme 2. R³=R⁶=—CH₃, and R⁸=R⁹=—H)

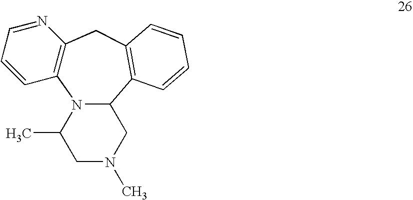

Synthesis of the title compound can be accomplished in an identical manner to the synthesis of the ligand described in Example 5 except that N¹-t-Boc-1-phenyl-1,2-diamino-ethane is replaced with N¹-t-Boc-1-N²-methyl-1,2-diaminoethane in Step 1. All other R³ and R⁴ substituted derivatives can be prepared in an identical manner using other α-bromoacid chlorides and N¹-t-Boc-1-N²-alkyl-1,2-diaminoethane.

### EXAMPLE 7

### Synthesis of Ligand 27. (Scheme 2. R¹ =—CH3. and R³=R⁸=R⁹=—H)

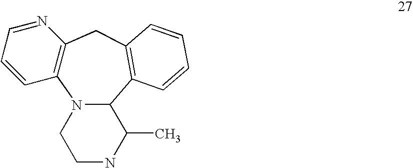

Step 1. A biphasic mixture of 3-amino-2-benzylpyridine (20 mmol) in methylene chloride (50 mL) and sodium carbonate (30 mmol) in water (50 mL) is vigorously stirred and cooled to 10-15° C. in ice bath. Then, 2-bromopropionyl chloride (22 mmol) is then added dropwise to the mixture at such a rate that the internal temperature is maintained at about 15° C. After the addition, the stirring is continued at ambient temperature for about 2 hours. Thereafter, the organic layer is separated, washed with 5% hydrochloric acid, and water. The organic phase is then dried over magnesium sulfate, filtered, and the filtrate evaporated in vacuo to give 3—N—(bromoacetyl)amino-2-benzyl-pyridine.

Step 2. A solution of the bromo derivative (10 mmol) from Step 1 and phosphoros oxychloride (12 mmol) in anhydrous, ethanol-free chloroform (20 mL) is stirred and heated under reflux for 16 hours. After cooling, the reaction mixture is poured onto saturated bicarbonate solution. The organic layer is then separated, washed with brine, dried over sodium sulfate, filtered, and the filtrate evaporated in vacuo to give the crude imine, which is purified by chromatography or recrystallization.

Step 3. A solution of the imine (10 mmol) from Step 2 in ethanol (15 mL) is treated with sodium borohydride (10 mmol) and stirred at ambient temperature for 4 hours. The reaction mixture is poured onto water, and extracted with methylene chloride. The organic layer is then separated, washed with brine, dried over sodium sulfate, filtered, and the filtrate evaporated in vacuo (at room temperature) to give the crude amine, which is used immediately for the next step in order to prevent intramolecular alkylation.

Step 4. A biphasic mixture of the amine from Step 3 (20 mmol) in methylene chloride (50 mL) and sodium carbonate (30 mmol) in water (50 mL) is vigorously stirred and cooled to 10-15° C. in ice bath. Then, 2-bromoacetylchloride (21 mmol) is then added dropwise to the mixture at such a rate that the internal temperature was maintained at about 15° C. After the addition, the stirring is continued at ambient temperature for about 2 hours. Thereafter, the organic layer is separated, washed with 5% hydrochloric acid, and water. The organic phase is then dried over magnesium sulfate, filtered, and the filtrate evaporated in vacuo to give the crude dibromide, which is purified by chromatography or recrystallization.

Step 5. A mixture of the dibromide (10 mmol) from Step 2, and ammonium carbonate (20 mmol) in glyme (20 mL) is stirred and heated under reflux for 6 hours. Thereafter, the reaction mixture is filtered hot and the filtrate evaporated in vacuo to give the crude lactam, which is purified by chromatography or recrystallization.

Step 6. A solution of the lactam derivative (10 mmol) from Step 5 in anhydrous tetrahydrofuran (20 mL) is stirred and cooled to 0° C. under inert atmosphere. Thereafter a solution of lithium aluminium hydride (1M in THF, 10 mL) is carefully added. After the addition, the solution is heated under reflux for 4 hours. Thereafter, the reaction mixture is cooled to 0° C. under inert atmosphere and very carefully treated with water added dropwise to decompose excess reducing agent. After complete decomposition, the solution was treated with anhydrous sodium sulfate, kept at ambient temperature for 1 hour and filtered. The filtrate is evaporated in vacuo to give the crude ligand 27, which is purified by chromatography or recrystallization.

### EXAMPLE 8

### Synthesis of Ligand 28, (Scheme 2. R¹=R³=—CH₃, and R⁸=R⁹=—H)

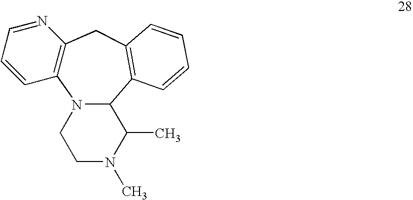

Synthesis of the title compound can be accomplished in an identical manner to the synthesis of the ligand described in Example 7 except that ammonium carbonate is replaced by methylamine in Step 4. All other R³ and R⁴ substituted derivatives can be prepared in an identical manner using other a-bromoacid chlorides in Step 1 and any primary alkylamines or anilines in Step 4.

### REFERENCES

1. Van Wijngaarden, I et al. The concept of selectivity in 5-HT receptor research. Eur. J. Pharmacol. 1990, 188, 301-312.
2. Peroutka, S. J. and Snyder, S. H. Multiple serotonin receptors: Differential binding of [³H]5-hydroxytryptamine, [³H]lysergic acid diethylamide, [³H]spiroperidol, Mol. Pharmacol. Rev. 1979, 16, 687-699.
3. Copeland, A. L. and Sorensen, J. L. Differences between methamphetamine users and cocaine users in treatment. Drug Alcohol Depend. 2001, 62, 91-95.
4. (McMahon, L. R. et al. Differential regulation of of the mesoaccumbens circuit by serotonin 5-hydroxytryptamine (5-HT)2A and (5-HT)2C receptors. J. Neurosci. 2001, 21, 7781-7787).
5. Tran-Nguyen, L. T. et al. Serotonergic depletion attenuates cocaine-seeking behavior in rats. Psychopharmacology (Berl) 1999, 146, 60-66.
6. Schama, K. F. et al. Serotonergic modulation of the discriminative-stimulus effects of cocaine in squirrel monkeys. Psychopharmacology (Berl) 1997, 132, 27-34.
7. Munzar, P. et al. Effects of various serotonin agonists, antagonists, and uptake inhibitors on the discriminative-stimulus effects of methamphetamine in rats. J. Pharmacol. Exp. Ther. 1999, 291, 239-250).
8. Satel, H. L. et al. Tryptophan depletion and attenuation of cue-induced craving for cocaine. Am. J. Psychiatry 1995, 152, 778-783.
9. Braumann, M. H. et al. Alterations in serotonergic responsiveness during cocaine withdrawal in rats: similarities to major depression in humans. Society of Biological Psychiatry 1998, 44, 578-591.
10. Davidson, C. et al. 5-H₂ receptor antagonists given in the acute withdrawal from daily cocaine injections can reverse established sensitization. Eur. J. Pharmacol. 2002, 255-263.
11. Nestler, E. J. Molecular basis of long-term plasticity underlying addiction. Nat. Rev. Neurosci. 2001, 2, 119-128.
12. van der Burg, W. J. Tetracyclic compounds. U.S. patent 1977; U.S. Pat. No. 4,062,848.
13. S. M. Berge, S. M. et al. J. Pharmaceutical Sciences 1977, 66, 1-end.
14. T. Higuchi and V. Stella. Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

We claim:

1. A compound of Formula 4, 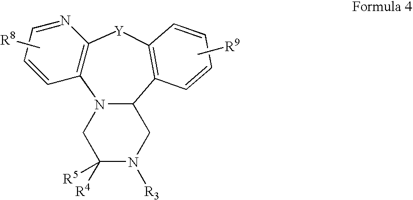
wherein X is —CH or —N—; Y is selected from the group consisting of —CR¹⁰R¹¹, —NR¹², —O—, —S—, —SO—, and —SO₂—; R³ and R¹² are independently selected from the group consisting of hydrogen; C₁-C₁₀ alkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; R⁴is selected from the group consisting of C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; R⁵, R¹⁰, and R¹¹ are independently selected from the group consisting of hydrogen, C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and R⁸ and R⁹ are independently selected from the group consisting of hydrogen; C₁-C₁₀ alkyl, hydroxyl, C₁-C₁₀ alkoxyl, C₁-C₁₀ hydroxyalkyl, amino, C₁-C₁₀ alkylamino, C₁-C₁₀ dialkylamino, carboxyl, and C₁-C₁₀ alkxoylcarbonyl.

2. The compound of claim 1, wherein X is —CH or —N—; Y is selected from the group consisting of —CR¹⁰R¹¹, —NR¹², —O—, and —S—; R³ and R¹² are independently selected from the group consisting of hydrogen, C₁-C₁₀ alkyl, phenyl, halophenyl, hydroxyphenyl, methoxyphenyl, benzyl, hydroxybenzyl, methoxybenzyl, and halobenzyl; R⁴ is selected from the group consisting of C₁-C₁₀ alkyl, phenyl, halophenyl, hydroxyphenyl, methoxyphenyl, benzyl, hydroxybenzyl, methoxybenzyl, and halobenzyl; R⁵, R¹⁰, and R¹¹ are hydrogen; and R⁸ and R⁹ are hydrogen or hydroxyl.

3. The compound of claim 1, wherein X is —N—; Y is —CR¹⁰R¹¹; R³ is selected from the group consisting of hydrogen, C₁-C₁₀ alkyl, phenyl, and benzyl; R⁴ is selected from the group consisting of C₁-C₁₀ alkyl, phenyl, halophenyl, hydroxyphenyl, methoxyphenyl, benzyl, hydroxybenzyl, methoxybenzyl, and halobenzyl; R⁵, R¹⁰, and R¹¹ are hydrogen; and R⁸ and R⁹ are hydrogen or hydroxyl.

4. The compound of claim 1, wherein X is —N—; Y is —NR¹²; R³ and R¹² are independently selected from the group consisting of hydrogen, C₁-C₁₀ alkyl, phenyl, and benzyl; R⁴ is selected from the group consisting of C₁-C₁₀ alkyl, phenyl, halophenyl, hydroxyphenyl, methoxyphenyl, benzyl, hydroxybenzyl, methoxybenzyl, and halobenzyl; R⁵, R¹⁰, and R¹¹ are hydrogen; and R⁸ and R⁹ are hydrogen or hydroxyl.

5. The compound of claim 1, wherein X is —CH—; Y is —CR¹⁰R¹¹; R³ is selected from the group consisting of hydrogen, C₁-C₁₀ alkyl, phenyl, and benzyl; R⁴ is selected from the group consisting of C₁-C₁₀ alkyl, phenyl, halophenyl, hydroxyphenyl, methoxyphenyl, benzyl, hydroxybenzyl, methoxybenzyl, and halobenzyl; R⁵, R¹⁰, and R¹¹ are hydrogen; and R⁸ and R⁹ are hydrogen or hydroxyl.

6. A compound of Formula 5, 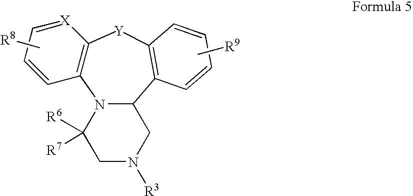
wherein X is —CH or —N—; Y is selected from the group consisting of —CR¹⁰R¹¹, —NR¹², —O—, —S—, —SO—, and —SO₂—; R³ and R¹² are independently selected from the group consisting of hydrogen; C₁-C₁₀ alkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; R⁶ is selected from the group consisting of C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; R⁷, R¹⁰, and R¹¹ are independently selected from the group consisting of hydrogen, C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and R⁸ and R⁹ are independently selected from the group consisting of hydrogen; C₁-C₁₀ alkyl, hydroxyl, C₁-C₁₀ alkoxyl, C₁-C₁₀ hydroxyalkyl, amino, C₁-C₁₀ alkylamino, C₁-C₁₀ dialkylamino, carboxyl, and C₁-C₁₀ alkxoylcarbonyl.

7. The compound of claim 6, wherein X is —CH or —N—; Y is selected from the group consisting of —CR¹⁰R¹¹, —NR¹², —O—, and —S—; R³ and R¹² are independently selected from the group consisting of hydrogen, C₁-C₁₀ alkyl, phenyl, halophenyl, hydroxyphenyl, methoxyphenyl, benzyl, hydroxybenzyl, methoxybenzyl, and halobenzyl; R⁶ is selected from the group consisting of C₁-C₁₀ alkyl, phenyl, halophenyl, hydroxyphenyl, methoxyphenyl, benzyl, hydroxybenzyl, methoxybenzyl, and halobenzyl; R⁷, R¹⁰, and R¹¹ are hydrogen; and R⁸ and R⁹ are hydrogen or hydroxyl.

8. The compound of claim 6, wherein X is —N—; Y is —CR¹⁰R¹¹; R³ is selected from the group consisting of hydrogen, C₁-C₁₀ alkyl, phenyl, and benzyl; R⁶ is selected from the group consisting of C₁-C₁₀ alkyl, phenyl, halophenyl, hydroxyphenyl, methoxyphenyl, benzyl, hydroxybenzyl, methoxybenzyl, and halobenzyl; R⁷, R¹⁰, and R¹¹ are hydrogen; and R⁸ and R⁹ are hydrogen or hydroxyl.

9. The compound of claim 6, wherein X is —N—; Y is —NR¹²; R³ and R¹² are independently selected from the group consisting of hydrogen, C₁-C₁₀ alkyl, phenyl, and benzyl; R⁴ is selected from the group consisting of C₁-C₁₀ alkyl, phenyl, halophenyl, hydroxyphenyl, methoxyphenyl, benzyl, hydroxybenzyl, methoxybenzyl, and halobenzyl; R⁵, R¹⁰, and R¹¹ are hydrogen; and R⁸ and R⁹ are hydrogen or hydroxyl.

10. The compound of claim 1, wherein X is —CH—; Y is —CR¹⁰R¹¹; R³is selected from the group consisting of hydrogen, C₁-C₁₀ alkyl, phenyl, and benzyl; R⁴ is selected from the group consisting of C₁-C₁₀ alkyl, phenyl, halophenyl, hydroxyphenyl, methoxyphenyl, benzyl, hydroxybenzyl, methoxybenzyl, and halobenzyl; R⁵, R¹⁰, and R¹¹ are hydrogen; and R⁸ and R⁹ are hydrogen or hydroxyl.

11. A compound of Formula 6, 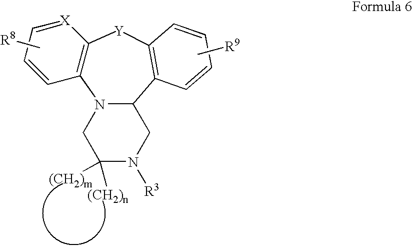
wherein m and n independently vary from 1 to 4; X is —CH or —N—; Y is selected from the group consisting of —CR¹⁰R¹¹, —NR¹², —O—, —S—, —SO—, and —SO₂—; R³ and R¹² are independently selected from the group consisting of hydrogen; C₁-C₁₀ alkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; R⁸ and R⁹ are independently selected from the group consisting of hydrogen; C₁-C₁₀ alkyl, hydroxyl, C₁-C₁₀ alkoxyl, C₁-C₁₀ hydroxyalkyl, amino, C₁-C₁₀ alkylamino, C₁-C₁₀ dialkylamino, carboxyl, and C₁-C₁₀ alkxoylcarbonyl; and R¹⁰ and R¹¹ are independently selected from the group consisting of hydrogen, C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl.

12. The compound of claim 11, wherein m and n independently vary from 1 to 4; X is —CH or —N—; Y is selected from the group consisting of —CR¹⁰R¹¹, —NR¹², —O—, and —S—; R³ and R¹² are independently selected from the group consisting of hydrogen, C₁-C₁₀ alkyl, phenyl, halophenyl, hydroxyphenyl, methoxyphenyl, benzyl, hydroxybenzyl, methoxybenzyl, and halobenzyl; R⁸ and R⁹ are hydrogen or hydroxyl; and R¹⁰ and R¹¹ are hydrogen.

13. The compound of claim 11, wherein m and n independently vary from 1 to 4; X is —N—; Y is —CR¹⁰R¹¹; R³is selected from the group consisting of hydrogen, C₁-C₁₀ alkyl, phenyl, and benzyl; R⁸ and R⁹ are hydrogen or hydroxyl; and R¹⁰ and R¹¹ are hydrogen.

14. The compound of claim 11, wherein m and n independently vary from 1 to 4; X is —N—; Y is —NR¹²; R³ and R¹² are independently selected from the group consisting of hydrogen, C₁-C₁₀ alkyl, phenyl, and benzyl; R⁸ and R⁹ are hydrogen or hydroxyl; and R¹⁰ and R¹¹ are hydrogen.

15. The compound of claim 11, wherein m and n independently vary from 1 to 4; X is —CH—; Y is —CR¹⁰R¹¹; R³ is selected from the group consisting of hydrogen, C₁-C₁₀ alkyl, phenyl, and benzyl; R⁸ and R⁹ are hydrogen or hydroxyl; and R¹⁰ and R¹¹ are hydrogen.

16. A compound of Formula 7, 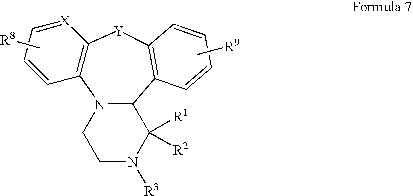
wherein X is —CH or —N—; Y is selected from the group consisting of —CR¹⁰R¹¹, —NR¹², —O—, —S—, —SO—, and —SO₂—; R³ and R¹² are independently selected from the group consisting of hydrogen; C₁-C₁₀ alkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; R¹ is selected from the group consisting of C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; R², R¹⁰, and R¹¹are independently selected from the group consisting of hydrogen, C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and R⁸ and R⁹ are independently selected from the group consisting of hydrogen; C₁-C₁₀ alkyl, hydroxyl, C₁-C₁₀ alkoxyl, C₁-C₁₀ hydroxyalkyl, amino, C₁-C₁₀ alkylamino, C₁-C₁₀ dialkylamino, carboxyl, and C₁-C₁₀ alkxoylcarbonyl.

17. A compound of claim 16, wherein X is —CH or —N—; Y is selected from the group consisting of —CR¹⁰R¹¹, —NR¹², —O—, and —S—; R³ and R¹² are independently selected from the group consisting of hydrogen, C₁-C₁₀ alkyl, phenyl, halophenyl, hydroxyphenyl, methoxyphenyl, benzyl, hydroxybenzyl, methoxybenzyl, and halobenzyl; R¹ is selected from the group consisting of C₁-C₁₀ alkyl, phenyl, halophenyl, hydroxyphenyl, methoxyphenyl, benzyl, hydroxybenzyl, methoxybenzyl, and halobenzyl; R², R¹⁰, and R¹¹ are hydrogen; and R⁸ and R⁹ are hydrogen or hydroxyl.

18. The compound of claim 16, wherein X is —N—; Y is —CR¹⁰R¹¹; R³ is selected from the group consisting of hydrogen, C₁-C₁₀ alkyl, phenyl, and benzyl; R¹ is selected from the group consisting of C₁-C₁₀ alkyl, phenyl, halophenyl, hydroxyphenyl, methoxyphenyl, benzyl, hydroxybenzyl, methoxybenzyl, and halobenzyl; R², R¹⁰, and R¹¹ are hydrogen; and R⁸ and R⁹ are hydrogen or hydroxyl.

19. The compound of claim 16, wherein X is —N—; Y is —NR¹²; R³ and R¹² are independently selected from the group consisting of hydrogen, C₁-C₁₀ alkyl, phenyl, and benzyl; R¹ is selected from the group consisting of C₁-C₁₀ alkyl, phenyl, halophenyl, hydroxyphenyl, methoxyphenyl, benzyl, hydroxybenzyl, methoxybenzyl, and halobenzyl; R², R¹⁰, and R¹¹ are hydrogen; and R⁸ and R⁹ are hydrogen or hydroxyl.

20. The compound of claim 16, wherein X is —CH—; Y is —CR¹⁰R¹¹; R³ is selected from the group consisting of hydrogen, C₁-C₁₀ alkyl, phenyl, and benzyl; R¹ is selected from the group consisting of C₁-C₁₀ alkyl, phenyl, halophenyl, hydroxyphenyl, methoxyphenyl, benzyl, hydroxybenzyl, methoxybenzyl, and halobenzyl; R², R¹⁰, and R¹¹ are hydrogen; and R⁸ and R⁹ are hydrogen or hydroxyl.

21. A method of performing a therapeutic procedure comprising administering and effective amount of a pharmaceutically acceptable compostion of Formula 4, 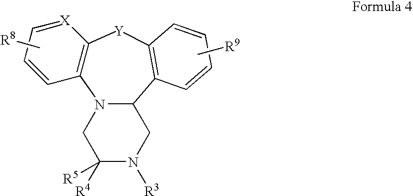
wherein X is —CH or —N—; Y is selected from the group consisting of —CR¹⁰R¹¹, —NR¹², —O—, —S—, —SO—, and —SO₂—; R³ and R¹² are independently selected from the group consisting of hydrogen; C₁-C₁₀ alkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; R⁴ is selected from the group consisting of C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; R⁵, R¹⁰, and R¹¹ are independently selected from the group consisting of hydrogen, C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and R⁸ and R⁹ are independently selected from the group consisting of hydrogen; C₁-C₁₀ alkyl, hydroxyl, C₁-C₁₀ alkoxyl, C₁-C₁₀ hydroxyalkyl, amino, C₁-C₁₀ alkylamino, C₁-C₁₀ dialkylamino, carboxyl, and C₁-C₁₀ alkxoylcarbonyl.

22. The method of claim 21, wherein the therapeutic procedure comprises treating patients having drug addiction.

23. The method of claim 21, wherein the therapeutic procedure comprises treating patients having anxiety.

24. The method of claim 21, wherein the therapeutic procedure comprises treating patients having depression.

25. A method of performing a therapeutic procedure comprising administering and effective amount of a pharmaceutically acceptable compostion of Formula 5, 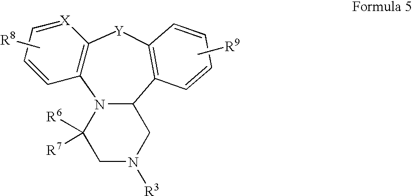
wherein X is —CH or —N—; Y is selected from the group consisting of —CR¹⁰R¹¹, —NR¹², —O—, —S—, —SO—, and —SO₂—; R³ and R¹² are independently selected from the group consisting of hydrogen; C₁-C₁₀ alkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; R⁶ is selected from the group consisting of C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; R⁷, R¹⁰, and R¹¹ are independently selected from the group consisting of hydrogen, C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and R⁸ and R⁹ are independently selected from the group consisting of hydrogen; C₁-C₁₀ alkyl, hydroxyl, C₁-C₁₀ alkoxyl, C₁-C₁₀ hydroxyalkyl, amino, C₁-C₁₀ alkylamino, C₁-C₁₀ dialkylamino, carboxyl, and C₁-C₁₀ alkxoylcarbonyl.

26. The method of claim 25, wherein the therapeutic procedure comprises treating patients having drug addiction.

27. The method of claim 25, wherein the therapeutic procedure comprises treating patients having anxiety.

28. The method of claim 25, wherein the therapeutic procedure comprises treating patients having depression.

29. A method of performing a therapeutic procedure comprising administering and effective amount of a pharmaceutically acceptable compostion of Formula 6, 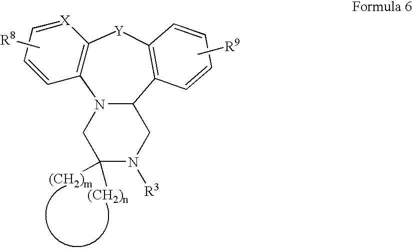
wherein m and n independently vary from 1 to 4; X is —CH or —N—; Y is selected from the group consisting of —CR¹⁰R¹¹, —NR¹², —O—, —S—, —SO—, and —SO₂—; R³ and R¹² are independently selected from the group consisting of hydrogen; C₁-C₁₀ alkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; R⁸ and R⁹ are independently selected from the group consisting of hydrogen; C₁-C₁₀ alkyl, hydroxyl, C₁-C₁₀ alkoxyl, C₁-C₁₀ hydroxyalkyl, amino, C₁-C₁₀ alkylamino, C₁-C₁₀ dialkylamino, carboxyl, and C₁-C₁₀ alkxoylcarbonyl; and R¹⁰ and R¹¹ are independently selected from the group consisting of hydrogen, C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl.

30. The method of claim 29, wherein the therapeutic procedure comprises treating patients having drug addiction.

31. The method of claim 29, wherein the therapeutic procedure comprises treating patients having anxiety.

32. The method of claim 29, wherein the therapeutic procedure comprises treating patients having depression.

33. A method of performing a therapeutic procedure comprising administering and effective amount of a pharmaceutically acceptable compostion of Formula 7, 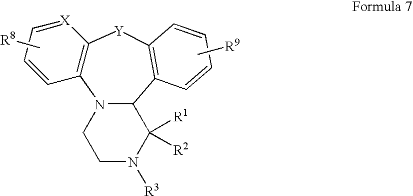
wherein X is —CH or —N—; Y is selected from the group consisting of —CR¹⁰R¹¹, —NR¹², —O—, —S—, —SO—, and —SO₂—; R³ and R¹² are independently selected from the group consisting of hydrogen; C₁-C₁₀ alkyl; C₅-C₁₀ aryl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, C₁-C₁₀ acyl, C₁-C₁₀ hydroxyalkyl, amino, C₁-C₁₀ alkylamino, C₁-C ₁₀ dialkylamino, and C₁-C₁₀ alkxoylcarbonyl; R¹ is selected from the group consisting of C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; R², R¹⁰, and R¹¹ are independently selected from the group consisting of hydrogen, C₁-C₁₀ alkyl; C₁-C₁₀ alkxoylcarbonyl; C₅-C₁₀ aryl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; C₅-C₁₀ arylalkyl unsubstituted or substituted with C₁-C₁₀ alkyl, hydroxyl, C₁-C₁₀ alkoxyl, cyano, halo, trihaloalkyl, carboxyl, amino, C₁-C₁₀ alkylamino, C₁-C₁₀ dialkylamino, and C₁-C₁₀ alkxoylcarbonyl; and R⁸ and R⁹ are independently selected from the group consisting of hydrogen; C₁-C₁₀ alkyl, hydroxyl, C₁-C₁₀ alkoxyl, C₁-C₁₀ hydroxyalkyl, amino, C₁-C₁₀ alkylamino, C₁-C₁₀ dialkylamino, carboxyl, and C₁-C₁₀ alkxoylcarbonyl.

34. The method of claim 33, wherein the therapeutic procedure comprises treating patients having drug addiction.

35. The method of claim 33, wherein the therapeutic procedure comprises treating patients having anxiety.

36. The method of claim 33, wherein the therapeutic procedure comprises treating patients having depression.
